(19) United States
(12) Patent Application Publication
(10) Pub. No.: US 2006/0100682 A1
(43) Pub. Date: May 11, 2006

(54) Ice belt to reduce body temperature

(76) Inventor: Shirley B. Koffroth, Palmdale, CA (US)

Correspondence Address: Mrs. Shirley B. Koffroth, 1342 Jaguar Court, Palmdale, CA, 93551, US

(21) Appl. No.: 11/298,191

(22) Filed: Dec. 8, 2005

Related U.S. Application Data
(63) Continuation-in-part of application No. 10/816,420, filed on Apr. 1, 2004.
(60) Provisional application No. 60/499,930, filed on Sep. 3, 2003.

(51) Int. Cl. A61F 7/00

(52) U.S. Cl. 607/114

(57) ABSTRACT

The Ice Belt To Reduce Body Temperature is a belt made of a turned out strip of non-elastic, pliable material of amble width and length to gird the waist from the midriff down, contains pocket-like receptacles on its innermost body-contacting side for the placement of flexible ice bags, and is secured around the waist by a connecting means consisting of mating Velcro fasteners. The Ice Belt to Reduce Body Temperature is a new concept of belts intended for sports, exercise and safety because the Ice Belt To Reduce Body Temperature provides the wearer with the comfort of a lower body temperature when exposed to high climatic conditions while participating in aerobic-type activity. In fact, most belts pertaining to the prior art are designed to promote sweating—a symptom of high body temperature—rather than reduce sweating. Therefore, the Ice Belt helps to lessen the risk of the user acquiring heat exhaustion when exposed to high climatic conditions.

### CROSS-REFERENCE TO RELATED APPLICATION

This is a Non-Provisional patent application claiming the benefit of U.S. Non-Provisional patent Ser. No. 10/816,420, filed Apr. 1, 2004 and incorporated by reference in its entirety. Said application: Ser. No. 10/816,420, having also claimed benefit of the U.S. Provisional Patent Application Ser. No. 60/499,930 filed Sep. 9, 2003.

### STATEMENT REGARDING FED SPONSORED R & D

There is no sponsoring.

### SEQUENCE LISTING A TABLE OR A COMPUTER PROGRAM APPENDIX

None

### DESCRIPTION

### BACKGROUND OF THE INVENTION

The Prior Art is replete with cooling devices that employ pocket-like receptacles for the placement of temperature transference agents. There are many therapeutic vest-like devices that cover the torso; mostly intended for curing wounds and lowering body temperature caused by biophysiologic conditions such as fever and multiple sclerosis: U.S. Pat. No. 5,484,448; No. 5,146,625; No. 3,950,789. There are also many cooling vests to help reduce heat stress induced by high temperatures in industrial facilities and the like: U.S. Pat. No. 5,146,625, No. 5,038,779, No. 2,748,391; as well as abundant ace-type bandage wraps that use this therapeutic method for medical treatment to cure injuries; mostly specified for direct contact with small body parts such as the finger, arm, ankle or knee: U.S. Pat. No. 6,582,383; No. 4,676,247; No. 5,484,448; No. 6,048,326, No. 4,556,055; No. 6,656,210. All complicated devices with multiple uses of hot/cold temperature transference. Admirable and conclusive for said purposes, nevertheless, none of the above mentioned devices address the problem of lowering body temperature for the safety of avoiding heat stress when the user is engaged in strenuous exercise under high, climatic conditions. Neither are the Prior Arts specifically designed to comfortably gird the waist; an area absent of vital organs and important nerve centers; thus reducing the risk of discomposure of the body's functions when confronted with ice. Nor do the Prior Art elements lend themselves to said usage as follows.

The above mentioned devices—of course not including the cooling vests, but taking particular notice of the elastic bandage wraps—are not only difficult to take on and off given their elastic property and internal connecting means, but also make replacing the temperature agents difficult to replenish given this procedure necessitates the intervention of a third party. Precisely because said devices are intended for as little aggravation as possible to a patient's wounds; to mention only a few of the problems inherent to the known types of temperature transference devices now present in the Prior Art

In view of the above mentioned disadvantages latent to the known types of temperature transference devices now present in the Prior Art, what is needed then to satisfy the above deficiencies, is a non-elastic cooling device designed exclusively to gird the waist; a body region that does not contain vital organs or excessive nerve centers. Said non-elastic cooling device of the present invention is a belt with ample lengths of press type hand-adjustable fasteners located on its extremities; thus accommodating waistband variables such as breathing, sitting, standing—more amply explained further on—by expanding or disbanding the Ice Belt. Something that the previously mentioned Prior Art's internal fasteners overlapping the devices upon themselves rather than the fasteners upon themselves, do not contain; thus making accommodating waistband variables impossible and the devices unsuitable to fulfill the intended purpose of the Ice Belt. That of lowering body temperature and avoiding heat exhaustion in high climatic temperatures when the user is engaged in aerobic type activity. Although it might be possible to employ an elastic bandage wrap on the waist to heal an injury, due to its limited stretch, it would be suffocating to gird the waist with an elastic wrap in high temperatures for the purpose of the present invention. In fact, the said Prior Arts are not specified for use on the waist.

Furthermore, the present invention sets itself apart because the Ice Belt, unlike the above mentioned Prior Arts, can be taken off in a matter of seconds for refreezing in its entity, and as such, can be quickly replaced with a freshly frozen belt; thus departing from standard procedures for conditioning and replacing temperature transference agents, one by one; the a forehand is treated more amply in the detailed description of the invention and its claims. Yet another enhancement of the present invention and its improvement over the Prior Art, is its easy removal due to the press type external fasteners. Another advantage of the present invention is the double thickness of the belt's non-elastic, pliable material allowing for a turned out measurement of sufficient length and width to comfortably gird the waist (see drawings, FIGS. 3 & 4) from the midriff down. Therefore, it is understood that the girding of the waist for the present invention's purpose—that of lowering body temperature and avoiding heat exhaustion when the user is exposed to high climatic conditions while engaged in aerobic-type activity such as sports, martial arts such as Toe Kwon Do, military activity and police work; over ones clothing and under an armored shield or bullet proof vest in the case if the latter three activities, not excluding other body exertions and work, inclusively, housework—must be accomplished with a double thickness of non-elastic, pliable fabric such as cotton. In fact, in the same situation the elastic bandage-type wraps of the Prior Art applied to the waist for the above stated purpose of the present invention, would bend, wrinkle, cause perspiration and in general, great discomfort.

The a forehand stated problems of the Prior Art are only a few that the present invention addresses and solves; the improvements treated in more detail in its brief summary and claims.

### BRIEF SUMMARY OF THE INVENTION

With the foregoing in mind, then it is understood that the general purpose of the present invention, which will be described subsequently in greater detail, is to provide the user of the Ice Belt with protection from the dangerous condition of heat exhaustion; preventive medicine as such and not the cure of a medical condition that already exists. To accomplish this, the present invention generally comprises two long strips of non-elastic pliable material for a turned out measurement of sufficient length and width to comfortably gird the waist from the midriff down; two ample lengths of mating, hand adjustable press type fasteners on its extremities, overlapping one upon the other, rather than upon the main body of the belt; the said mating belt fasteners long enough for easy hand adjustment to variables of the waistband's measurement; at least six consecutive pocket-like receptacles on the innermost body contacting side of the belt for the placement of ice bags—type blue or treated ice; a fastening means on the inner side of the upper portion of the pocket-like receptacles to secure the ice bags from falling out when placed around the waist. In conclusion, a non-stretchable cooling device exclusively designed to gird the waist; an area free of vital organs and central nerve centers. Thus, the present invention is a cooling device that does not require a time involving procedure to replace the temperature transference agents, that can be easily taken on and off, that can accommodate variables in the waistband measurements by simply opening its press-like closing means situated one to each side of its extremities, and readjusting them to a smaller or larger position; and that can be refrozen in its entity rather than replacing its ice packs one by one. Therefore, the present invention, while containing some of the former properties of the Prior Art such as the pocket-like receptacles for the placement of its cooling agents, has many novel features not found in the same; which renders it a temperature transference device that is not anticipated or obvious, either alone or in combination thereof.

### BRIEF DESCRIPTION OF THE DRAWINGS

FIG. 1 is a perspective view of the Ice Belt in extended condition showing all of it's components and drawn to a reduced scale.

FIG. 2 is a perspective view of the Ice Belt in curled configuration. The drawing is approximately the same scale as FIG. 1.

FIG. 3 is a front view and shows the Ice Belt fastened onto a dummy figure. FIG. 3 is drawn in a similar scale as FIGS. 1 & 2.

FIG. 4 is a back view and shows the Ice Belt fastened onto a dummy figure. FIG. 4 is drawn similar to the scales used in FIGS. 1, 2, & 3.

### DESCRIPTION OF THE PREFERRED EMBODIMENTS

Similar parts are designated by the same reference characters in the several views of my invention, the Ice Belt To Reduce Body Temperature, from now on referred to as, the Ice Belt.

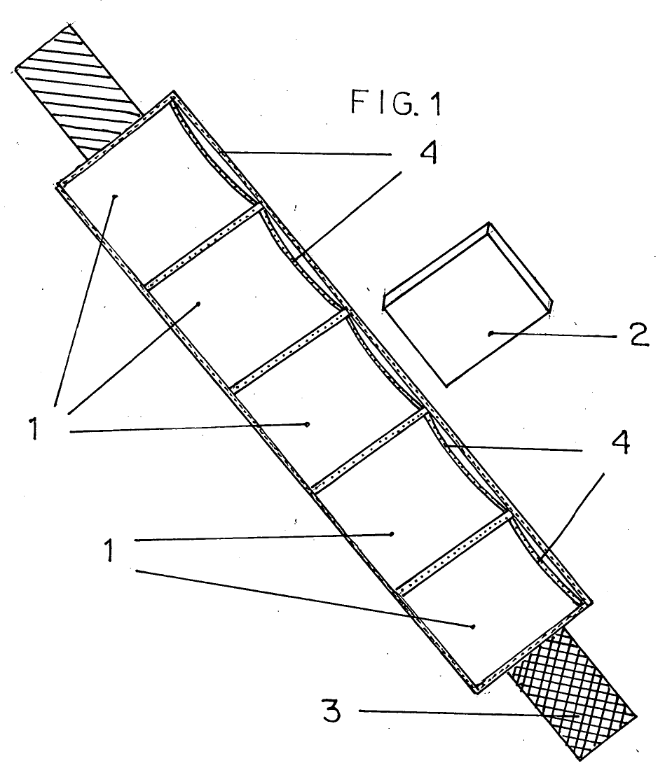

This invention, the Ice Belt, FIG. 1, incorporates the use of pocket-like receptacles, Ref. 1, for the insertion of flexible bags, Ref. 2, made of plastic and containing frozen blue or treated ice. The Ice Belt is attached at the ends by mating fasteners of Velcro, Ref 3. The pocket-like receptacles, Ref. 1, contain a connecting means of fasteners placed on the inner-top side, Ref. 4, of the pocket-like receptacles to secure the pocket.

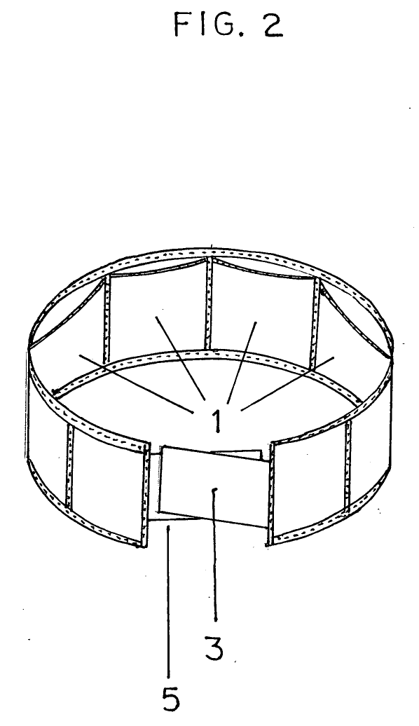

FIG. 2 is a perspective view of the Ice Belt in curled configuration. When the Ice belt is closed as shown in FIG. 2, the pockets containing the ice bags are located to the inner side of the belt, Ref. 1. FIG. 2 also shows the Velcro fasteners, Ref. 3, located on the belt's extremities, closed to their tightest position as the fasteners can be accommodated to a loose or tightened position, Ref. 5, depending on changes in the waistband measurements.

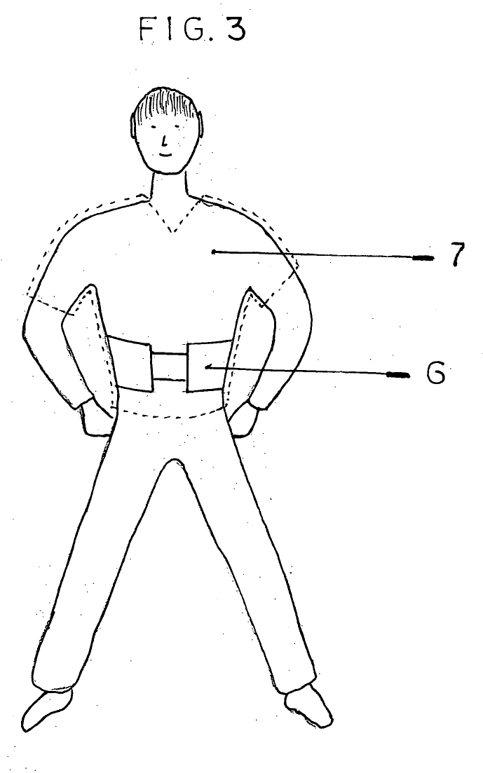

FIG. 3 is a front view and shows the Ice Belt, Ref. 6, fastened from the dummy figure's midriff down to his waist. The drawing in FIG. 3 also depicts the Ice Belt, Ref. 6, as shown underneath a body garment; a shirt, Ref. 7.

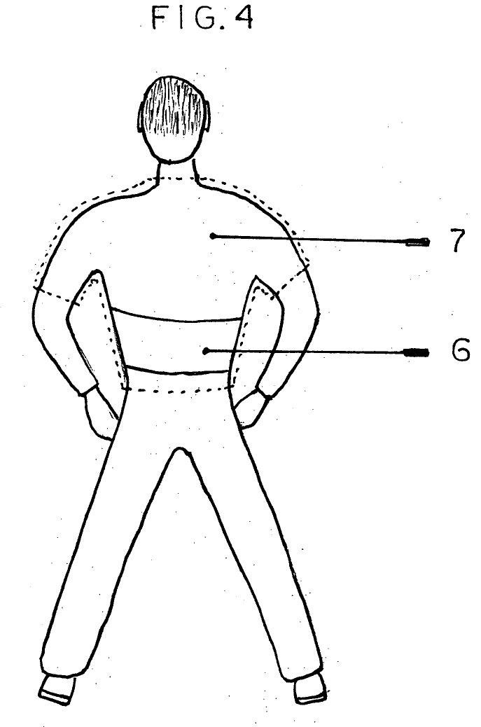

FIG. 4 shows the Ice Belt, Ref. 6, from behind as it looks when positioned on the wearer. The drawing in FIG. 4 also depicts the Ice Belt, Ref. 6, as shown underneath a body garment; in this case, a shirt, Ref. 7.

### DETAILED DESCRIPTION OF THE INVENTION

The invention—an Ice belt to reduce body temperature—consists of a strip of non-elastic material made of any pliable fabric approximately 4 to 5 inches wide and the length depending on the user's waist measurement. The Ice Belt is equipped with pocket-like receptacle sewn to the strip of non-elastic material on the innermost body-contacting side of the Ice Belt, the pocket-like receptacle's function is to hold flexible bags of blue or treated ice. The Ice Belt is designed to fasten around the midriff and extend to the waist by means of an ample length of Velcro sewn to each extremity of the Ice Belt. These Velcro fasteners on both ends of the Ice Belt's extremities should be of sufficient length for accommodation of normal changes in the waistband measurement. The above because a person's waistband measurement is not constant. It basically falls into seven different measurement length classifications: standing, before eating; standing, after eating; sitting, before eating and sitting after eating. Also there is one more variable regarding waistband measurement common to each of the seven measurement classifications, and this is the effect on waist measurement length caused from breathing, both inhaling and exhaling.

By the use of extended lengths of Velcro sewn to the Ice Belt's extremities; the Ice Belt can accommodate to expanding or disbanding conditions of the waistband simply by opening the Velcro fasteners sewn to the Ice Belt's extremities and readjusting the fasteners; thus accommodating the Ice Belt to a slightly smaller or larger waistband measurement.

Plus this suggested facility of extended lengths of Velcro for hand adjustment of waistband changes, the Ice Belt should be fabricated in various sizes; small, medium, large, extra large etc; the measurements depending on whether the Ice Belt is intended for a child, adolescent, man or woman, heavy or thin. The Ice Belt is more effective when worn underneath ones clothing with the pocket-like receptacles that are attached to the innermost body-contacting side of the Ice Belt in direct contact with the user's body. But is also effective when used over ones clothing with or without an armored shield or bullet proof vest.

The Ice Belt's front piece fabrication is very simple as it consists of sewing together two long strips of flexible material cut to allow hemming for a turned out measurement of from 4 to 5 inches in width, and the length depending on the desired waist measurement. The ends of the strip of non-elastic materials should be left open for the insertion of the Velcro fasteners; once the two sewn strips of flexible material have been turned out, the Velcro fasteners are sewn to both of the Ice Belt's extremities. Then the securing method (snaps or Velcro etc.) are placed to the inner-top side, Ref. 4, of the pocket-like receptacles. The strip of pockets, Ref. 1, is then secured to the Ice Belt's front piece section, by sewing it into place on the bottom and sides of the turned out strip of non-elastic material to its innermost body contacting side. The ice bags, Ref. 2, are frozen before placed inside the Ice Belt's receptacles. When the ice in the ice bags melts—depending on the temperature, body functions, and outer garment—used bags can be replaced by freshly frozen ones, or the Ice Belt in its entity can be put into a freezer compartment until the liquid in the bags contained in the Ice Belt's pockets has frozen again. In a temperature from about 85 F. to 95 F., the ice should stay in tack for about 2 to 3 hours, depending on body exertion and the outer garment. In higher temperatures, the ice may only last for an hour, also depending on the outer garment and the exertion used while wearing the Ice Belt. If worn under a bullet proof vest for military combat or police work, or an armored shield as used for Martial Arts—both insulating devices—the ice's duration can be doubled.

The present embodiments of this invention are to be considered in all respects as descriptive and illustrative, but not restrictive; the scope of the invention being indicated by the appended claims rather than by the foregoing description; therefore, any changes considered more functional to the original design and utility of my invention, the Ice Belt to Reduce Body Temperature, as long as they come within the meaning and range of my invention's basic conception, a device to lower body temperature and avoid heat exhaustion during aerobic-type activities under high climatic conditions, are intended to be embraced therein.

What I claim as my invention is:

1. An Ice belt to reduce body temperature and avoid heat exhaustion for sports, exercise & safety when the user is exposed to high climatic temperatures, comprising;
a. two long strips of non-elastic pliable material for a turned out measurement of sufficient length and width to comfortably gird the waist from the midriff down;
b. two ample lengths of mating, hand-adjustable press type fasteners on its extremities, overlapping one upon the other, rather than upon the main body of the belt;
c. the said mating belt fasteners long enough for easy hand adjustment to variables of the waistband's measurement;
d. at least six consecutive pocket-like receptacles on the innermost body contacting side of the belt for the placement of ice bags—type blue or treated ice;
e. a fastening means on the inner side of the upper portion of the pocket-like receptacles to secure the ice bags from falling out when placed around the waist.

2. A method of refreezing the Ice belt to reduce body temperature and avoid heat exhaustion for sports, exercise & safety when the user is exposed to high, climatic temperatures—defined as two long strips of non-elastic pliable material for a turned out measurement of sufficient length and width to comfortably gird the waist from the midriff down; two ample lengths of mating, hand-adjustable press type fasteners on its extremities, overlapping one upon the other, rather than upon the main body of the belt; said mating belt fasteners long enough for easy hand adjustment to variables of the waistband's measurement; at least six consecutive pocket-like receptacles with fastening means on the innermost body contacting side of the belt for the placement of type blue or treated ice bags—in its entity, rather than refreezing the ice bags and replacing them one by one; comprising the steps of:
a. removing the Ice belt from the user;
b. rolling the Ice belt into a small compact unit; and
c. placing the unit in its entity into a freezer compartment.

3. A method for non-interrupted protection from heat exhaustion with the Ice belt to reduce body temperature as recited in claim 2, comprising the steps of;
a. removing the melted Ice belt from the user by quickly pulling loose from the waist the ample lengths of mating, hand-adjustable press type fasteners located on the Ice belt's extremities;
b. taking a freshly frozen Ice belt from the freezer compartment and girding the waist with it;
c. pressing shut the mating, press-like fasteners located on the Ice belt's extremities.

4. A method of employing the Ice belt to reduce body temperature as recited in claim 2, for replacing the ice bags, one by one, in their pocket-like receptacles, comprising the steps of:
a. removing the Ice belt from the user's waist;
b. taking out the used ice bags from their receptacles; and
c. putting freshly frozen ice bags into the pocket-like receptacles.

5. A method of wearing the Ice belt, as recited in claim 2, over ones clothing; comprising the steps of:
a. taking a freshly frozen Ice belt from its freezer compartment;
b. comfortably securing the two ample lengths of mating, hand-adjustable press type fasteners on its extremities, around the waist over ones clothing, then overlapping them upon themselves to the disbanding or expanding waistband measurement coinciding with the existing waistband variables.

6. The use of the Ice belt to reduce body temperature and avoid heat exhaustion for sports, exercise & safety when the user is exposed to high climatic temperatures—defined as two long strips of non-elastic pliable material for a turned out measurement of sufficient length and width to comfortably gird the waist from the midriff down; two ample lengths of mating, hand-adjustable press type fasteners on its extremities, overlapping one upon the other, rather than upon the main body of the belt; the said mating belt fasteners long enough for easy hand adjustment to variables of the waistband's measurement; at least six consecutive pocket-like receptacles on the innermost body contacting side of the belt for the placement of ice bags—type blue or treated ice; a fastening means on the inner side of the upper portion of the pocket-like receptacles to secure the ice bags from falling out when placed around the waist—associated with sports, armored shields and bullet proof vests.

7. The use of the Ice belt as recited in claim 6 associated with military activities, over ones clothing and under a bullet proof vest.

8. The use of the Ice belt as recited in claim 6 associated with police work, over ones clothing and under a bullet proof vest.

9. The use of the Ice belt as recited in claim 6 associated with football.

10. The use of the Ice belt as recited in claim 6, over ones clothing and under an armored shield, associated with the martial arts such as Toe Kwon Do.

11. The use of the Ice belt as recited in claim 6 associated with basketball.

12. The use of the Ice belt as recited in claim 6 associated with baseball.

13. The use of the Ice belt as recited in claim 6 associated with Track (running).

14. The use of the Ice belt as recited in claim 6 associated with Soccer Football.

15. The use of the Ice belt as recited in claim 6 associated with bicycling.

16. The use of the Ice belt as recited in claim 6 associated with tennis.

17. use of the Ice belt as recited in claim 6 associated with aerobics.

18. The use of the Ice belt as recited in claim 6 associated with all outdoor sports.

19. The use of the Ice belt as recited in claim 6 associated with all indoor sports.

20. The use of the Ice belt as recited in claim 6 associated with, but not limited to, normal everyday activities.
